(19) United States
(12) Patent Application Publication
(10) Pub. No.: US 2008/0255049 A1
(43) Pub. Date: Oct. 16, 2008

(54) COMBINED USE OF ULTRASOUND AND GROWTH FACTORS TO STIMULATE BONE FORMATION

(75) Inventors: Dale Richman Sumner, Chicago, IL (US); Amarjit Singh Virdi, Chicago, IL (US); Robert Maynard Leven, Chicago, IL (US)

Correspondence Address: BARNES & THORNBURG LLP, P.O. BOX 2786, CHICAGO, IL, 60690-2786, US

(73) Assignee: RUSH UNIVERSITY MEDICAL CENTER, Chicago, IL (US)

(21) Appl. No.: 12/101,031

(22) Filed: Apr. 10, 2008

Related U.S. Application Data
(60) Provisional application No. 60/911,068, filed on Apr. 10, 2007.

(51) Int. Cl. A61K 38/18; A61P 19/00

(52) U.S. Cl. 514/12

(57) ABSTRACT

The combination of low intensity pulsed ultrasound with administration of an osteogenic protein or proteins or their genes enhances bone repair in mammals. This combination avoids thermal activation of tissue repair mechanisms and allows the osteogenic protein or proteins or their genes to act at the site of bone fracture or lowered bone density to enhance or accelerate bone formation, thereby improving the prognosis of a patient. The combination may be used to treat fractures, to prevent or treat osteoporosis, to improve outcomes for bone replacement and to prevent or treat bone loss due to other physiologic disorders.

### PRIORITY CLAIM

This application claims priority to copending U.S. provisional application 60/911,068 filed Apr. 10, 2007.

### BACKGROUND

Although bone repair often occurs without complication, there are a significant number of cases in which intervention may improve healing, including delayed fracture repair, skeletal trauma, primary or revision arthroplasty, spinal arthrodesis, and bone tumour resection. These needs are often met with autologous bone grafting which has drawbacks associated with risks of a second surgery, neurological morbidity, increased blood loss, prolonged anesthesia, infection, longstanding pain and cosmetic deformity at the excision site. In addition, autografting may be ineffective if the amount of graft material needed exceeds the amount of bone available for grafting. Allograft is an alternative to autograft. However, some of the same risks are present with added limitations including excessive resorption, transmission of disease, or specific host immune responses. Other approaches to stimulate bone growth are the use of bone graft substitutes, growth factors and physical modalities such as ultrasound. Growth factors approved by FDA for bone repair clinically include recombinant human bone morphogenetic protein-2 (rhBMP-2) and rhBMP-7.

Diseases and injuries associated with bone and cartilage have a significant impact on the population. Approximately five million bone fractures occur annually in the United States alone. About 10% of these have delayed healing and of these, 150,000 to 200,000 nonunion fractures occur accompanied by loss of productivity and independence. Fractured bones heal readily in the young, but more slowly in the elderly. In all cases the time that is required for even common uncomplicated fractures to heal results in considerable morbidity, loss of time from work and inconvenience. Hence, a need exist for agents that might lead to more rapid healing of bone fractures at all ages.

Directly related to bone fracture is osteoporosis, a family of diseases characterized by net loss of bone mass. In Europe, Japan and the U S., an estimated 75 million people are thought to be effected by the disease including an estimated ten million persons in the United States. In the United States, almost 34 million more are thought to have low bone mass. One in two women and one in four men over age 50 will have an osteoporosis-related fracture in their remaining life. Osteoporosis is responsible for almost two million fractures annually, including: hip fractures, vertebral fractures, wrist fractures and fractures at other sites. Osteoporosis-related bone loss leads to significant health care cost mostly by predisposing people to fractures and pain originating from defective bone remodeling in response to physical stress.

Bone remodeling is the fundamental and highly integrated process of resorption and formation of bone tissue that results in precisely balanced skeletal mass with renewal of the mineralized matrix. This renewable process is achieved without compromising the overall anatomical architecture of bones. This continuous process of internal turnover ensures that bone maintains a capacity for true regeneration and maintenance of bone integrity by continuous repairing of microfractures and alterations in response to stress. The architecture and composition of the adult skeleton is in perpetually dynamic equilibrium. Remodeling also provides a means for release of calcium in response to homeostatic demands. Conditions that influence bone remodeling include mechanical stimuli such as immobilization or weightlessness, electric current or electromagnetic fields such as capacitively coupled electric field or pulsed electromagnetic field, hormonal changes or in response to certain inflammatory diseases.

Bone remodeling occurs through orchestrated cycles of activity that include activation, resorption, reversal, formation, and quiescence steps. Activation is characterized by the existence of a thin layer of lining cells. Then circulating mononuclear cells of hematopoetic lineage begin to migrate into the activation site and fuse together to form osteoclasts. Activation is followed by resorption where active osteoclasts excavate a bony surface. This step typically lasts about 2-4 weeks. Reversal occurs following resorption and continues for a period of 9 days. During this time inactive pre-osteoblasts are present in the resorption depressions. The next step is formation and takes about 3-4 months. During this stage active osteoblasts refill the excavation site. The last phase of bone remodeling is quiescence where no remodeling activity occurs until the beginning of the next remodeling cycle. Ideally the quantity of bone fill must equal the quantity resorbed with no loss of bone mass.

Bone is thus constantly being remodeled through bone resorption by osteoclasts and bone formation by osteoblasts. When these two processes are not balanced, and bone resorption is greater than bone formation, then osteoporosis results. After age 30 bone mass begins to decrease in both males and females. However after estrogen production decreases in menopausal woman the imbalance between bone formation and bone resorption becomes more pronounced and bone loss becomes more rapid.

In addition to bone loss due to aging and changes in hormone balance during menopause, patients treated with steroids also develop osteoporosis. Bone loss is also important in other conditions such as acute and chronic renal failure, hyperparathyroidism, Paget's disease and periodontal disease. This list of alternate conditions represents a subset of the diseases related to bone loss and should not be considered limiting.

In recent years, various attempts have been made to stimulate bone growth. Mammalian bone tissue is known to contain multiple proteins, presumably active during growth and natural bone healing, which can induce a developmental cascade of cellular events resulting in new bone formation. This active factor (or factors) has variously been referred to in the literature as bone morphogenic proteins, bone inductive proteins, osteogenic proteins, osteoinductive proteins.

Many osteogenic proteins belong to a family of proteins known as the transforming growth factors-beta (TGF-beta) super family of proteins and include the bone morphogenetic proteins (BMPs), activins and inhibins. More than thirty members belong to the TGF-beta-super-family. The BMP-family is divided into subfamilies including the BMPs, such as BMP-2 and BMP-4, osteogenic proteins (OPs), such as OP-1 or BMP-7, OP-2 or BMP-8, BMP-5, BMP-6 or Vgr-1, cartilage-derived morphogenetic proteins (CDMPs), such as CDMP-1 or BMP-14 or GDF-5, growth/differentiation factors (GDFs), such as GDF-1, GDF-3, GDF-8, GDF-9, GDF-11 or BMP-11, GDF-12 and GDF-14, and other subfamilies, such as BMP-3 or osteogenin, BMP-9 or GDF-2, and BMP10. Local delivery of osteogenic proteins or genes that express osteogenic proteins induce bone growth and repair in mammals. The subfamily of proteins known generally as BMPs has been reported to have osteogenic activity, and other growth and differentiation type activities. Combined treatment of a bone defect with rhBMP-2 and rhTGF-beta2 had an additive effect on bone healing in an in vivo canine model. Some combinations of growth factors are more effective than use of a single growth factor to stimulate bone healing or bone formation in vivo. Combined treatment of a bone defect with rhBMP-2 and rhTGF-beta2 had an additive effect on bone healing in an in vivo canine model. Other effective combinations include platelet-derived growth factor (PDGF) combined with insulin-like growth factor-1 (IGF-1), IGF-1 combined with TGF-beta-1, bmp-2 combined, insulin like growth factor-1 combined with transforming growth factor-beta-1, BMP-2 combined with BMP-3 and TGF-beta, TGF-beta1 combined with BMP-2, TGF-beta2 combined with BMP-2 and BMP-3, TGF-beta1 combined with BMP-7, TGF-beta3 combined with BMP-2. Vascular endothelial growth factor (VEGF), PDGFs, and fibroblast growth factors (FGFs) are also known to stimulate in vivo bone healing. Small molecules such as active fragments of the growth factors or other small peptides that have osteogenic activity such as TP-508 have also been reported to stimulate bone repair.

Ultrasound (US) is sound with a frequency greater than the upper limit of human hearing, this limit being approximately 20 kilohertz. Ultrasound is used widely in medical imaging and therapeutic modalities and is considered generally benign because it does not use ionizing radiation. Ultrasound energy has two physiological effects in the body: it enhances the inflammatory response and it can heat soft tissue. Ultrasound energy has been reported accelerate bone growth during fracture healing and distraction osteogenesis, possibly by stimulating expression of osteogenic growth factor genes.

Low-intensity pulsed ultrasound (LIPUS) is an ultrasound method for applying non-thermal, low intensity ultrasound energy to a patient. LIPUS has been reported to accelerate fracture healing in both animal models and clinical trials, but the complete mechanisms of action remain unclear.

Bone fracture, bone loss and osteoporosis remain serious medical and economic problems despite current understanding of the fundamental cellular components of bone remodeling and the molecules that underlie bone resorption. Thus, there is a need for improved methods to treat fractured bones more effectively and to prevent and treat bone loss due to aging, osteoporosis and other disorders.

### SUMMARY

Addition of ultrasound treatment to growth factors, growth factor fragments or small molecules used solely or in combination provides synergistic stimulation for bone formation.

Methods and compositions for stimulating osteogenesis in a human or an animal after an injury or disease has caused a fracture or weakening of the bone, are disclosed. More specifically the method combines the local or systemic delivery of a composition comprising an osteogenic factor or factors or genes, with ultrasound treatment to improve bone formation and repair after injury or to prevent or treat reduced bone density due to disease. The compositions and methods are used to promote fracture healing and skeletal repair and acceleration of fracture healing.

For example, the combination of low intensity pulsed ultrasound (LIPUS) with an osteogenic protein (rhBMP-2) increased bone formation and accelerated repair. Two methods to improve bone repair include the use of recombinant human bone morphogenetic protein-2 (rhBMP-2) and low-intensity pulsed ultrasound (LIPUS). A well-characterized ectopic implant model was used to determine if LIPUS enhances the effect of rhBMP-2. Absorbable collagen sponges loaded with 0, 1, 2.5 or 5 μg doses of rhBMP-2 were implanted subcutaneously in 11 week old, male Long Evans rats, followed by daily 20 minute sham or active LIPUS treatment beginning 1 day after surgery. At 2 weeks, LIPUS had no effect on rhBMP-2 induced bone formation as assessed by micro computed tomography (μCT), but at 4 weeks, active LIPUS increased bone volume in the 1 μg rhBMP-2 treated implants 117.8-fold (p=0.028), and 2.3-fold in the 5 μg dose implants (p=0.077) compared to sham LIPUS. Histologic staining for mineralized tissue was consistent with the μCT observations. LIPUS enhances bone formation induced by rhBMP-2 and this effect is synergistic because LIPUS by itself did not stimulate bone formation in this model. Many LIPUS signals when combined with known or suspected osteogenic proteins or genes would have a similar synergistic effect on bone formation.

LIPUS enhanced rhBMP-2 induced ectopic bone formation. Variable doses of rhBMP-2 were loaded onto collagen sponges and implanted subcutaneously in rats with subsequent sham or active LIPUS treatment. Low doses of rhBMP-2 were intentionally used to avoid maximizing the biological response to the growth factor. A statistically significant enhancement by LIPUS on rhBMP-2 induced ectopic bone formation was found at the 1 μg dose and a positive effect observed with the 5 μg rhBMP-2 dose.

Sub-dermal rhBMP-2 loaded collagen sponges were chosen because this represents a well characterized model of bone formation that allowed simple application of LIPUS. The results from “control” group (sham LIPUS) were similar to previous findings in the literature.

As disclosed herein, BMP-2 delivered via a synthetic carrier was shown previously to have a dose dependent effect on ectopic bone formation.

Although bone volume and the total mineral content increased with addition of LIPUS treatment, the mineral density of the bone volume was relatively unchanged or slightly decreased with the application of LIPUS. Thus, since more total bone was detected, the process of mineralization did not occur at a faster rate by the application of LIPUS and it may have been slightly slower.

Growth factors peptides fragments and derivatives of growth factors, growth factor analogs, synthetic growth factors and derivatives thereof are suitable. For example, if used systemically, carriers such as calcium phosphate, calcium sulfate can be used. If used locally, growth factors may also be coated directly on an implant. Growth factors and analogs or derivatives thereof may also be formulated in a time-release or sustained release formulations. In addition, any stabilizing formulation or those that enhance delivery of growth factors in vivo can also be used.

The term consisting essentially of includes a composition that includes one or more growth factor that is effective therapeutically.

### BRIEF DESCRIPTION OF THE DRAWINGS

FIG. 1. Three-Dimensional Reconstructions of the Implants. Representative PCT images of varying rhBMP-2 doses loaded on absorbable collagen sponges after 4-weeks of treatment with either sham or active LIPUS treatment. The figure shows the micro computed tomography results from the Ectopic Bone Formation example disclosed herein White bar is 1 mm in length.

FIG. 2. Box plot of bone volume at varying rhBMP-2 doses at 4 weeks. The dark horizontal line indicates the median, the box extends from the 25th to the 75th percentile, and the whiskers indicate the largest and smallest observed values. These graphs depict the results of the quantitative analysis of the microcomputed tomography data from the Ectopic Bone Formation example disclosed herein.

FIG. 3. Photomicrographs at 4 weeks from the Ectopic Bone Formation example disclosed herein. Representative samples after 4-weeks of sham LIPUS and active LIPUS treatment at the 3 rhBMP-2 doses tested along with the 0 μg control. In each column, the left panels show the toluidine blue staining and the right panels show the von Kossa staining, a commonly used marker of mineralization in histology (horizontal black bar is 2 mm).

### DETAILED DESCRIPTION

LIPUS is used in combination with at least one osteogenic factor or factors to improve the overall growth of bone in a patient in need thereof. In a rat model, the effective dosage of rhBMP-2 ranges from 1 microgram to 5 micrograms. Although higher doses of rhBMP-2 were not tested, others have reported no further increase in bone formation in the model with higher doses so this seems sufficient for rats. In canines, doses of rhBMP-2 in the range of 25 micrograms to 800 micrograms were effective in a bone defect model. The dose of rhBMP-2 to stimulate bone repair is dependent upon species and anatomic site.

BMP-2 and other growth factors (e.g., TGF-beta) have a broad effective dose range when applied locally. Ultrasound enhances the bone formation capability over a majority of the growth factor dose range. Dosages for other growth factors to be used in combination with BMP-2 and ultrasound are expected to also fall into the range of 1 microgram to 10 milligrams when applied locally. Because growth factors disclosed herein act in similar fashion, BMP-2 results are predictive.

The combination of osteogenic factors and LIPUS improves the overall bone growth and repair substantially. The growth factors can be applied topically or systemically. The preferred application is topical for fractures and preferably systemic for disorders that cause loss of bone matrix such as osteoporosis. Growth factors or their genes which could be used alone or in combination include a family of proteins known as the transforming growth factors-beta (TGF-beta) super family of proteins and include the bone morphogenetic proteins (BMPs), activins and inhibins. More than thirty members belong to the TGF-beta-super-family. The BMP-family is divided to subfamilies including the BMPs, such as BMP-2 and BMP-4, osteogenic proteins (OPs), such as OP-1 or BMP-7, OP-2 or BMP-8, BMP-5, BMP-6 or Vgr-1, cartilage-derived morphogenetic proteins (CDMPs), such as CDMP-1 or BMP-14 or GDF-5, growth/differentiation factors (GDFs), such as GDF-1, GDF-3, GDF-8, GDF-9, GDF-11 or BMP-11, GDF-12 and GDF-14, and other subfamilies, such as BMP-3 or osteogenin, BMP-9 or GDF-2, and BMP10. Vascular endothelial growth factor (VEGF), PDGFs and fibroblast growth factors (FGFs) are also known to stimulate in vivo bone healing. Small molecules such as active fragments of the growth factors or other small peptides that have osteogenic activity such as TP-508 have also been reported to stimulate bone repair. The use of many of these factors has been shown in various models to improve bone formation; their use in combination with LIPUS will result in a synergistic improvement in bone formation.

Local delivery of single or multiple growth factors or genes is preferably done as described in Example 1 or by using any appropriate delivery vehicle as is standard in the art, including but not limited to collagen sponges, calcium phosphate mixtures, hydrogels, demineralized bone matrix or any other suitable biomaterial.

Peptides and fragments of the osteogenic factors or genes may also be used in place of the parent compound. Combinations of these fragments or genes or combinations of one or more fragments with one or more parent molecules are also suitable.

Suitable doses of growth factors including BMP-2 include for example, 1 μg, 2 μg, 3 μg, 5 μg, 10 μg, 15 μg, 25 μg, 50 μg, 100 μg, 200 μg, 500 μg or 1 mg. Other suitable ranges include, for example, 1-10 μg, 1-100 μg, 100-1,000 μg and 1 mg-10 mg. The dosage may also depend on factors such as the anatomical site. If a particular site is large, higher doses such as 10-100 mg may be used.

### EXAMPLE 1

### Ectopic Bone Formation Model in Rats

This model has been used for over 40 years to assess factors affecting bone formation. It is considered a standard assay to demonstrate that a growth factor, peptide, peptide fragment or gene is able to induce bone formation. It is considered one of several models for evaluating candidate materials for use in humans.

Absorbable collagen sponges (ACS) treated with 0 μg, 1 μg, 2.5 μg, or 5 μg of recombinant human BMP-2 (rhBMP-2) were implanted subcutaneously at one of four locations dorsally in 16 Long Evans male 11 week old rats. The rats received either daily low intensity pulsed ultrasound or sham treatment over the site of ACS implantation for 2 or 4 weeks, at which time the bone volume for each sample was determined by microcomputed tomography.

The ultrasound signal used in the experiment had an operating frequency of 1.5 MHz, an intensity of 30 mW/cm², and was applied for 20 minutes per day, 7 days per week.

The rhBMP-2 was applied directly to the absorbable collagen sponge in 200 micro liters of buffer.

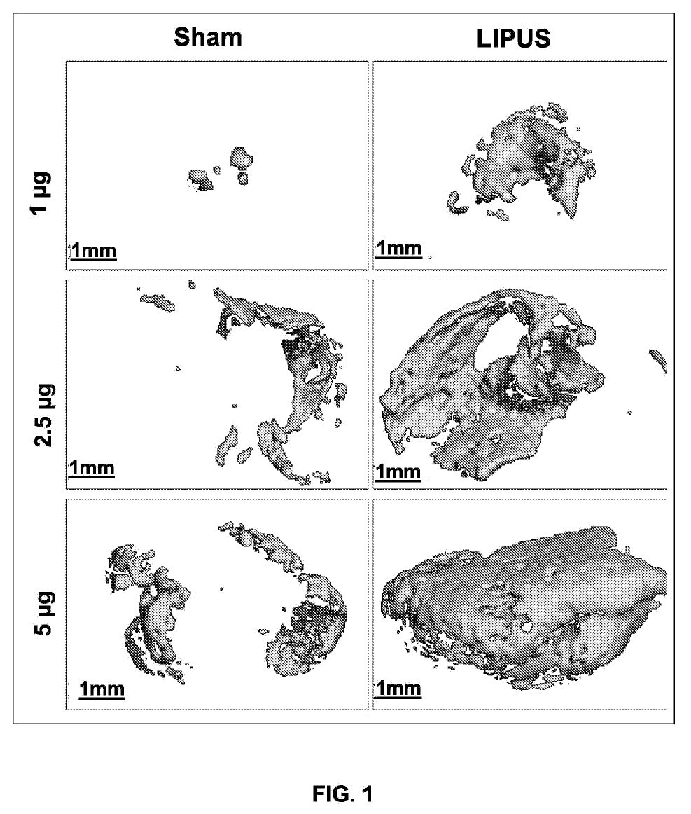

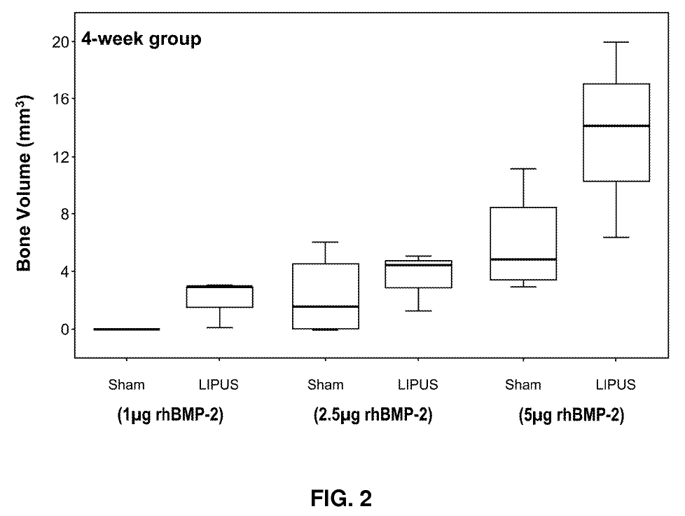

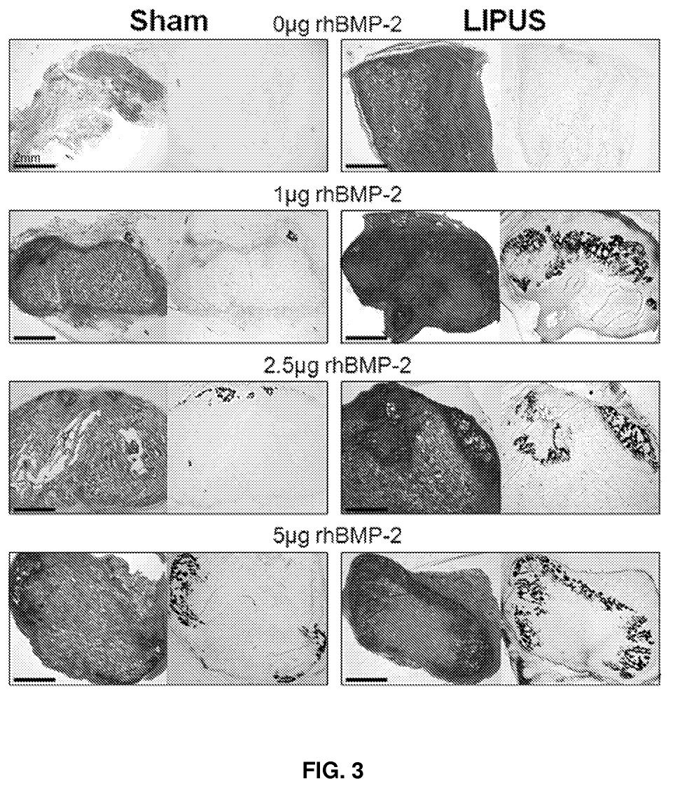

At 4 weeks, the low intensity pulsed ultrasound treatment increased the amount of bone that formed in the rhBMP-2 treated sponges (FIG. 1), 117.8-fold in the 1 μg rhBMP-2 group (p=0.028) and by 2.3-fold in the 5 μg dose group (p=0.077) (FIG. 2). These differences were not apparent at two weeks. FIG. 3 shows that endochondral bone was formed in the model. These results demonstrate that low intensity pulsed ultrasound enhances rhBMP-2 induced bone formation in a nonosseous ectopic environment. There were significant weight gains in each group from baseline to sacrifice (p<0.001), and the increases in body weight were not significantly different between the sham and active LIPUS groups at either 2 weeks (p=0.248) or 4 weeks (p=0.564).

The difference between groups appeared to be most prominent at the lowest rhBMP-2 dose as 3 of 8 implants with 1 μg rhBMP-2 with sham LIPUS treatments at 2 and 4 weeks had bone, whereas 6 of 7 implants at this same dose with active LIPUS treatment had bone (p=0.057, chi-square).

Bone volume as determined by μCT was similar in the sham and active LIPUS groups at 2 weeks, but was higher in the active LIPUS treated implants than in the sham LIPUS treated implants at 4 weeks (FIG. 2). The difference at 4 weeks was significant in the 1 μg group (p=0.028) and approached significance in the 5 μg group (p=0.077). These differences represented 117.7-fold and 2.3-fold increases in the mean bone volume, respectively. The mineral density of the bone was not affected by LIPUS treatment. Total mineral content (bone volume*local mineral density) followed the same pattern as bone volume, with the LIPUS effect significant at 4 weeks in the 1 μg group (p=0.028).

Bone volume increased significantly from 2 weeks to 4 weeks in the 5 μg rhBMP-2 doses of both the sham LIPUS (p=0.021) and active LIPUS (p=0.032) groups and also increased in the 2.5 μg dose of the active LIPUS group (p=0.034). Mineral density increased in the 2.5 μg dose of the active LIPUS group from 2 to 4 weeks (p=0.034) with a similar trend in the 5 μg dose of the active LIPUS group (p=0.083). Total mineral content increased from 2 to 4 weeks in the 5 μg dose of the sham LIPUS (p=0.021) and the 2.5 μg and 5 μg doses of the active LIPUS groups (p=0.034 and 0.032, respectively) with a similar trend in the 1 μg dose of the active LIPUS group (p=0.077).

Histological evaluation confirmed the presence of mineralized tissue as observed with μCT (FIG. 3). Implants not treated with rhBMP-2 lacked von Kossa staining, a well-accepted marker of mineralization. In the rhBMP-2 treated implants, mineralized tissue was occasionally observed at 2 weeks, but was more patterned and prominent at 4 weeks. In the 4 week rhBMP-2 treated implants, mineralized tissue was noted mostly on the periphery of the implants and addition of active LIPUS treatment increased the amount of mineralized tissue.

### EXAMPLE 2

Growth factors or their genes in combination with LIPUS provide a synergistic improvement in bone healing. It is anticipated that sites to be tested would include spinal fusion (where rhBMP-2 doses are in the range of 5 to 10 milligrams) and recalcitrant tibial fractures, in which similar quantities of rhBMP-2 are used. The carrier for the growth factor or genes (most likely rhBMP-2 or rhBMP-7, but potentially including a variety of known or newly discovered osteogenic proteins) could be a collagen sponge, a calcium phosphate or sulfate mixture, demineralized bone matrix, hydrogel or any other suitable biomaterial. LIPUS could range from 1 to 400 mW/cm².

### Materials and Methods

Animals, Surgery and Necropsy. Sixteen Long Evans male rats (Charles River Labs, Portage, Mich.) aged 11 weeks were used in the study in an IACUC approved study. The animals were housed in a photoperiod-controlled environment (temperature, 21° C., humidity, 50%, 12 h: 12 h light/dark cycle) and provided standard rat chow and water ad libitum throughout the study. All 16 animals in this study underwent the same surgical procedure. Animals were anesthetized with ketamine (100 mg/kg, intramuscularly) and xylazine (5 mg/kg, intramuscularly), placed in a prone position and the dorsal surface was shaved. A template was placed over the rat and an alcohol resistant surgical pen was used to mark the sites of sponge placement and to ensure consistent ultrasound transducer placement throughout the study. The dorsal surface was scrubbed with betadine and the surgical site was exposed through a sterile drape. Two 1.5-cm long incisions (1 cm from the midline, and thus 2 cm from each other) were made equidistant to the 4 marked template dots to expose the subcutaneous space for insertion of sponges. Blunt dissection was used to create 2 separate pockets at each incision site. With 2 incisions, 4 sponges were spaced evenly on the dorsal side of the rat (2 on the left of the spine and 2 on the right). Each rat received 4 sponges, with each sponge treated with one of the 4 doses of rhBMP-2 (0, 1, 2.5, or 5.0 μg) and placed in a separate sub dermal pocket. The sponge locations were systematically assigned so that the cranial-caudal distribution of doses was equalized. Absorbable 4-0 Vicryl sutures (Ethicon, Inc., Somerville, N.J.) were used to approximate the soft tissues at the incision site. Subcutaneous injection of buprenex analgesia (0.02 mg/kg diluted with saline) was given once for postoperative pain management. At the 2 or 4 week time points the rats were euthanized using carbon dioxide (CO₂) inhalation. A large midline incision was made to allow for in situ implant visualization and harvesting. Implants were carefully separated from the surrounding tissue, wrapped in gauze soaked in saline and frozen at −20° C.

rhBMP-2 Loading Onto Absorbable Collagen Sponges. Absorbable type I bovine collagen sponges (Helistat; Integra LifeSciences, Plainsboro, N.J.) were used as the rhBMP-2 carrier. The dimensions of the unloaded sponges were 12.5 mm by 12.5 mm by 5 mm, comparable to prior published experiments. Sponges were loaded under sterile conditions with 200 μl solution (sterile 5 mM glutamate, 5 mM NaCl, 2.5% glycine, 0.5% sucrose, 0.01% Tween-80 at a pH 4.5) containing 0, 1, 2.5, or 5.0 μg rhBMP-2 (Wyeth Research, Cambridge Mass.). After 1 hour at room temperature, the sponges were stored overnight in covered tissue culture plates at 4° C. and implanted the next day.

Low-Intensity Pulsed Ultrasound Treatment. Animals were treated daily for 2 weeks (8 rats, 4 active and 4 sham LIPUS) or for 4 weeks (8 rats, 4 active and 4 sham LIPUS) starting 1 day after surgery, using 3% isoflurane gas (Surgivet/Anesco Isotec 4) as an anesthetic. Ultrasonic coupling gel (Aquasonic 100 Ultrasound Transmission Gel, Parker Laboratories, Inc New Jersey) was placed on the skin overlying the 4 implantation sites and LIPUS was administered using a sonic accelerated fracture healing system device (THM Model 2 A, Collimage Type, Smith and Nephew Inc). Two Velcro straps were used to anchor the 4 ultrasound transducers. The ultrasound signal consisted of a 1.5-MHz sine wave frequency delivered in bursts lasting 200 μs followed by a pause of 800 μs with a frequency of 1 KHz. The power output was 30 mW/cm². LIPUS treatment was for 20 minutes each day. Sham treated animals were handled the same as treatment animals, except the power to the LIPUS generator was not turned on.

Microcomputed Tomography (μCT). Prior to μCT analysis the implants were placed in 10% neutral buffered formalin. Specimens were held securely in the manufacturer's 30.7 mm specimen holder which was filled with 10% neutral buffered formalin. The scanning parameters were 45 kV energy (X-ray voltage), 177 μA intensity, and 150 ms integration time with a 1024×1024 reconstructed image, yielding 30 μm inplane resolution with 30 μm thick slices (μCT-40, Scanco Medical, Wayne, Pa.). The reconstruction algorithm filtered noise and segmented the data set at a threshold of 200, using the manufacturer's software to calculate the bone volume (mm³) and mineral density of the bone volume (mg/cc of hydroxyapatite, based on a standard calibration device provided by the manufacturer). In addition, the total mineral content was calculated by multiplying bone volume with mineral density.

Histology. Fixed samples were, embedded in JB-4 (Polysciences, Inc., Warrenville, Pa.), and cut into 5 μm sections (Leica Microsystems, Model RM2255). Alternating sections were stained with toluidine blue O or with von Kossa (American Master Tech Scientific, Inc., Lodi, Calif.) counterstained with Nuclear Fast Red.

Statistical Analysis. Non-parametric statistical tests were used, including chi square to test for the presence/absence of bone, Mann-Whitney to test for group and time differences, Friedman to test for rhBMP-2 dose effects and Wilcoxon to compare the animal weight at baseline and sacrifice (SPSS Inc. Chicago, Ill.).

1. A method to enhance bone repair in a mammal in need thereof, the method comprising:
(a) selecting at least one osteogenic growth factor or its gene that stimulates bone growth;
(b) selecting parameters for ultrasound exposure that stimulate bone growth; and
(c) administering at least one selected factor and ultrasound, wherein the ultrasound is characterized by the selected parameters, to the mammal close enough in temporal proximity to interact in bone repair effects.

2. The method of claim 1 wherein the mammal is a human.

3. The method of claim 1 wherein the at least one factor is selected from the group consisting of BMP-2; rhBMP-2; BMP-4; osteogenic proteins (Ops), such as OP-1 or BMP-7, OP-2 or BMP-8, BMP-5, BMP-6 or Vgr-1; cartilage-derived morphogenetic proteins (CDMPs), such as CDMP-1 or BMP-14 or GDF-5; growth/differentiation factors (GDFs), such as GDF-1, GDF-3, GDF-8, GDF-9, GDF-11 or BMP-11, GDF-12 and GDF-14; and other subfamilies, such as BMP-3 or osteogenin, BMP-9 or GDF-2, BMP10, PDGF, IGF, VEGF, FGFs, periostin and/or any combinations thereof.

4. The method of claim 3 wherein at least the factor of rhBMP-2 is selected.

5. The method of claim 1 wherein the parameters selected for ultrasound exposure comprise a 1.5-MHz sine wave frequency delivered in bursts lasting 200 μs followed by a pause of 800 μs with a frequency of 1 KHz, with a power output of 30 mW/cm² for 20 minutes per day.

6. The method of claim 1 wherein the at least one factor is administered within 24 hours to 6 months of the ultrasound exposure.

7. The method of claim 1 wherein the mammal in need thereof has a condition selected from the group consisting of a fracture, non-unions, bone defect repair, osteoporosis, trauma, primary or revision dental implants, spinal arthrodesis, bone tumor resection, distraction osteogenesis, and combinations or variations thereof.

8. A method of increasing local bone formation during bone repair, the method comprising:
(a) providing a therapeutically effective amount of a growth factor;
(b) administering a low-intensity pulsed ultrasound (LIPUS) treatment;
(c) increasing bone formation amount during bone repair

9. The method of claim 1, wherein the parameters selected for ultrasound exposure comprise a power output from 1 to 400 mW/cm².

10. The method of claim 9, wherein the parameters for ultrasound exposure comprise a power output of 30 mW/cm2.

11. The method of claim 9, wherein the parameters for ultrasound exposure comprise a 1.5-MHz sine wave frequency.

12. The method of claim 10, wherein the parameters for ultrasound exposure comprise a 1.5-MHz sine wave frequency.

13. The method of claim 9, wherein the parameters for ultrasound exposure comprise application of a predetermined frequency of ultrasound in bursts lasting 200 μs followed by a pause of 800 μs with a frequency of 1 KHz.

14. The method of claim 10, wherein the parameters for ultrasound exposure comprise application of a predetermined frequency of ultrasound in bursts lasting 200 μs followed by a pause of 800 μs with a frequency of 1 KHz.

15. The method of claim 9, wherein the parameters for ultrasound exposure comprise application of the ultrasound for 20 minutes per day.

16. The method of claim 10, wherein the parameters for ultrasound exposure comprise application of the ultrasound for 20 minutes per day.
